(19) United States
(12) Patent Application Publication
(10) Pub. No.: US 2025/0130212 A1
(43) Pub. Date: Apr. 24, 2025

(54) METHOD AND DEVICE FOR FAST QUANTITATIVE DETERMINATION OF SEED TREATMENT LOADING

(71) Applicant: SYNGENTA CROP PROTECTION AG, Basel (CH)

(72) Inventor: David WITMER, Stein (CH)

(73) Assignee: SYNGENTA CROP PROTECTION AG, Basel (CH)

(21) Appl. No.: 18/834,286

(22) PCT Filed: Jan. 24, 2023
(86) PCT No.: PCT/EP2023/051664

§ 371 Date: Jul. 30, 2024

(30) Foreign Application Priority Data
Jan. 31, 2022 (EP) 22154338.2

(51) Int. Cl. G01N 30/95; G01N 21/64; G01N 33/00

(52) U.S. Cl. CPC G01N 30/95; G01N 21/6428; G01N 33/0098

(57) ABSTRACT

The present invention relates to a method for determining the presence and quantity of one or more agrochemically active ingredient(s) on the surface of one or more plant seed(s) or in a composition used or intended to be used to treat seed(s), comprising the steps as defined in claim 1.

### FIELD OF THE INVENTION

The present invention relates to a method and a device for the direct determination and quantitative analysis of multiple agrochemically active ingredients, such as active ingredients and growth promoters, especially on seeds. More particularly it relates to methods and devices for determining quantities of multiple active ingredients extracted especially from seeds of crop plants.

### BACKGROUND OF THE INVENTION

A reliable determination of agrochemically active ingredient composition, and the quantities thereof is very important in the agricultural and environmental practice. In agriculture, agrochemically active ingredients are employed in a variety of ways, including treating seeds by coating and/or immersing to protect them against many pests, including insects and soil borne pathogens, in particular using mixtures of different active ingredients.

It is particularly important to control the quantity of active ingredients coated on an individual seed because too little active ingredient will result in incomplete protection of the seed and emerging plant while too much active ingredient may have negative effects on the seed and its germination. A further disadvantage of excessive coating of active ingredients on seeds is unnecessary leaching of the active ingredient, or ingredients into the environment.

The term “active ingredient seed loading”, therefore, refers to the quantity of adhered active ingredient on the seed.

The loading of seed with agrochemically active ingredients can today be assessed qualitatively and quantitatively in a properly equipped analytical chemical laboratory. In a typical analytical chemical method for seed loading analysis, the active ingredients are extracted with solvent from the treated seed and the active ingredients in the resultant solution are separated and quantified using a high-performance liquid chromatograph (HPLC) equipped with an ultraviolet absorbance detector. Alternative laboratory-based detection methods such as infra-red spectrometry, mass spectrometry and gas chromatography (GC) coupled with flame-ionization detection may be used for particular active ingredients, but are generally less accurate. The disadvantage of the laboratory-based analysis methods is that they are technically demanding and require an appropriate equipment, leading to considerable capital cost to establish. This limits the availability of laboratory analyses and leads to a slow response time. A sample first needs to be shipped to a laboratory and be analysed there, which can take several days to weeks. The access and shipping times to an analytical laboratory is more difficult in rural settings, where seed production and treatment typically occur, and in developing countries. Laboratory methods such as HPLC are difficult to use outside of a laboratory environment, such as in the field, because the equipment parts are sensitive and fragile, and not designed to be translocated regularly.

Accordingly, there is a need for a method that can be performed locally, without having to send samples to laboratories, and without delay.

These limitations of the state-of-the-art methods are now overcome by the present invention through a number of experiments which ultimately provided successful results with seed analysis of commercially important seed treatment active ingredients from crop seeds such as cereals (e.g. barley, maize (corn), millet, oats, rye, sorghum, rice, wheat), soybean, cotton, sunflower and canola and oil seed rape under field conditions.

The loading for one or more active ingredient(s) for a batch of seeds can be determined for each active ingredient based on a reproducible chromatographic separation and reliable analytical data analysis at real time, and locally. Accordingly, the present invention therefore provides an indication of how well seeds are treated with a seed treatment composition.

### SUMMARY OF THE INVENTION

The present invention provides methods and devices for the quantitative analysis of multiple active ingredients, especially from a single seed or an aliquot of seeds or from a composition used or intended to be used to treat seed(s).

Accordingly, in a first aspect the present invention relates to a method for determining the presence and quantity of one or more active ingredient(s), more particularly one or more agrochemically active ingredient(s),
on one or more plant seed(s), and preferably on the surface of one or more plant seed(s), or
in a composition used or intended to be used to treat seed(s),
comprising the steps of:
   a. contacting the one or more plant seed(s) or said composition, with an extractant to extract one or more active ingredient(s), to yield a test sample;
   b. depositing a defined aliquot of the optionally filtered test sample on the starting line of a thin layer chromatographic (TLC) plate comprising a stationary phase coated on a substrate;
   c. optionally, depositing an aliquot of one or more reference compound(s), and preferably depositing an aliquot of a solution containing one or more reference compound(s), at a parallel starting position on the stationary phase; and optionally, drying the thus prepared thin layer chromatographic plate;
   d. contacting the thin layer chromatographic plate with a mobile solvent phase at the lower edge of the stationary phase in an elution chamber, and allowing the sample to elute for a predetermined period of time;
   c. removing the eluted thin layer chromatographic plate from the chamber, and optionally, drying the eluted thin layer chromatographic plate;
   f. positioning the eluted thin layer chromatographic plate in a detector unit equipped with an illumination source and an imaging device;
   g. taking at least one image of the illuminated thin layer chromatographic plate to detect the one or more signal(s) generated by the one or more active ingredient(s) and/or reference compound(s);
   h. matching a retention factor (Rf) of each detected signal to an expected Rf of a known active ingredient(s), and/or to a Rf of the reference compound(s); and
   i. quantifying the intensity of each signal to estimate the amount of the one or more specific active ingredient(s), preferably quantifying the intensity of fluorescence-quenching of each signal; and, optionally,
   j. extrapolating the determined ingredient quantity to the weight or number of the one or more seed(s) or said composition, subjected to extraction in step a., to obtain an active ingredient loading data, and preferably to obtain a seed active ingredient loading data.

The method according to the present invention can be used in a field, as field method, and/or can be used in different areas wherein active ingredients are used, and preferably wherein agrochemically active ingredients are used. In particular, the method according to the present invention can be related to one or several steps in the life cycle of treated seeds.

Said composition used or intended to be used to treat seed(s) (in which the presence and quantity of one or more active ingredient(s) can be determined) can be for example a seed treatment product, a slurry and/or a coating residue. Said composition can be used in the manufacturing of seed treatment products and/or in the commercialization of treated seeds, and can be found for example on the surface of the packaging or in the packaging used to contain treated seeds, on the surface of mixing tanks or machinery used for seed treatment, . . . etc.

In a second aspect, the present invention relates to the use of a mobile imaging device for an in-field use of the subject method.

In a further aspect, the present invention relates to a computer implemented method for determination and quantification of active ingredient signals on an eluted TLC plate from a raw image obtained according to the present invention, comprising the following steps:
a. detection of pixel regions in the raw image that contain the chromatographic signals;
b. assignment of the chromatographic signals to either extracted active ingredient or reference compound, and preferably to either extracted active ingredient or reference compound solution;
c. integration of the intensity of each signal in the pixel regions, said integration step c) comprising the following steps d) to g):
   a. converting RGB channels in the raw image into a luminance graph comprising a Y-channel;
   b. summing up pixel Y-intensities along the vertical dimension;
   c. detecting start-and end-points of peaks related to a signal; and
   d. integrating peak Y-intensities between peak start-and end-point; and
a. calculating active ingredient concentration by correlating the integrated peak areas of samples with the integrated peak areas of one or more reference compound(s).

In a further aspect, the present invention relates to a portable apparatus for determination and quantification of an active ingredient extracted from a seed or from a composition used or intended to be used to treat seed(s).

More particularly, said portable apparatus can comprise:
a. a housing comprising a first portion that prevents a thin layer chromatographic plate from moving in the housing portion, such that optionally the portion of the surface does not touch any portion of the first housing portion;
b. an illumination source positioned in the first housing; and
c. a second housing portion for fitting with the first housing portion and for engaging with an imaging device such that the device permits capture of an image under illumination;
wherein the apparatus is configured to permit optimal image acquisition conditions by excluding incandescent light and by having a defined geometry (relative position, e.g. angle and distance) for both illumination and image acquisition of a developed TLC plate.

In a further embodiment, said portable apparatus can comprise:
x1) a housing preventing a thin layer chromatographic plate from moving in said housing, such that optionally the portion of the surface does not touch any portion of the first housing portion;
y1) an illumination source positioned in said housing; and
z1) an imaging device engaged with said housing such that the device permits capture of an image under illumination,
wherein the apparatus is configured to permit optimal image acquisition conditions by excluding incandescent light and by having a defined geometry (relative position, e.g. angle and distance) for both illumination and image acquisition of a developed TLC plate.

In a further embodiment, said portable apparatus can comprise:
x2) a housing comprising a first portion that prevents a thin layer chromatographic plate from moving in said housing, such that optionally the portion of the surface does not touch any portion of the first housing portion;
y2) a second housing portion for fitting with the first housing portion and wherein an illumination source is positioned; and
z2) an imaging device engaged with said second housing portion such that the device permits capture of an image under illumination,
wherein the apparatus is configured to permit optimal image acquisition conditions by excluding incandescent light and by having a defined geometry (relative position, e.g. angle and distance) for both illumination and image acquisition of a developed TLC plate.

In a further aspect, the present invention relates to a kit of parts, especially for field use, comprising the apparatus according to the invention, a volume of the extractant, a volume of the mobile phase, thin layer chromatographic plates, reference standards and micro glass capillaries with a predefined capillary volume; and a mobile device comprising camera; and installed thereon, a software application.

### SHORT DESCRIPTION OF THE FIGURES

FIG. 1 shows an image taken from a UV-illuminated TLC plate, Herein, two rows of reference points indicating increasing concentrations of two active ingredients are shown.

FIG. 2 shows a luminance graph of the 2D-plate region containing the signals of one active ingredient (below), and a 1D-representation of the same spots where luminance intensities are summed up vertically (above). The integrated peak areas show the intensity of the fluorescence quenching, which can hence be correlated to the amount of compound in each signal spot.

### DETAILED DESCRIPTION OF THE INVENTION

The present invention is described in more detail below.

In a first aspect the present invention relates to a method, especially a field method, for determining the presence and quantity of one or more agrochemically active ingredient(s),
on one or more plant seed(s), and preferably on the surface of one or more plant seed(s), or
in a composition used or intended to be used to treat seed(s),
comprising the steps of:
   a. contacting the one or more plant seed(s) or said composition, with an extractant to extract one or more active ingredient(s), to yield a test sample;
   b. depositing a defined aliquot of the optionally filtered test sample on the starting line of a thin layer chromatographic (TLC) plate comprising a stationary phase coated on a substrate;
   c. optionally, depositing an aliquot of one or more reference compound(s), and preferably depositing an aliquot of a solution containing one or more reference compound(s), at a parallel starting position on the stationary phase; and optionally, drying the thus prepared thin layer chromatographic plate;
   d. contacting the thin layer chromatographic plate with a mobile solvent phase at the lower edge of the stationary phase in an elution chamber, and allowing the sample to elute for a predetermined period of time;
   c. removing the eluted thin layer chromatographic plate from the chamber, and optionally, drying the eluted thin layer chromatographic plate;
   f. positioning the eluted thin layer chromatographic plate in a detector unit equipped with an illumination source and an imaging device;
   g. taking at least one image of the illuminated thin layer chromatographic plate to detect the one or more signal(s) generated by the one or more active ingredient(s) and/or reference compound(s);
   h. matching a retention factor (Rf) of each detected signal to an expected Rf of a known active ingredient(s), and/or to a Rf of the reference compound(s); and
   i. quantifying the intensity of each signal to estimate the amount of the one or more specific active ingredient(s), preferably quantifying the intensity of fluorescence-quenching of each signal; and, optionally,
   j. extrapolating the determined ingredient quantity to the weight or number of the one or more seed(s) or of said composition, subjected to extraction in step a., to obtain an active ingredient loading data.

Offering all advantages of portable methods such as robust use under field conditions, a short response time (<30 min), technical complexity manageable by trained non-experts and low analysis costs, the present invention offers the attractive feature of simultaneous detection and quantification of multiple active ingredients directly, as do the lab methods using HPLC described above. This is achieved by a reproducible and robust chromatographic separation using thin layer chromatography (TLC) and utilization of a new combination of detection techniques coupled to automated image analysis at real time, and locally.

Accordingly, the present invention therefore provides an indication of how well seeds are treated with seed treatment product(s), at the location of treatment in a simple and reproducible manner involving low cost and robust equipment and a short response time.

Thin-layer chromatography (TLC) is known as a method for separating and detecting specific compounds from mixtures. The separation of a component by TLC is detected, for example, based on a difference in optical response between the separating agent layer and the detection target component, by visualizing signals or bands produced by the separation of compounds in a sample.

Generally, the method according to the invention comprises the steps of selecting an aliquot of seeds, extracting the active ingredients from the seed or seeds using a suitable extractant, optionally selected based on the active ingredient and seed being extracted, separation by TLC, and determination of an Rf value indicative of the eluted distance, to qualify the active ingredient, and the measurement of light intensity of the “spot” (the area of the TLC plate containing the active ingredient) and calculation of the integrated spot intensity, to determine the quantity of the active ingredient. One advantage of such a method is that it obviates the need for a filtration step between the extraction step and the chromatographic separation step.

The method is applicable to determination of a broad range of active ingredients when loaded on seeds of key agronomic crops such as, cereals (e.g. barley, maize (corn), millet, oats, rye, sorghum, rice, wheat), soybean, cotton, sunflower and canola and oil seed rape. Suitable crop seeds to be analysed in accordance with the invention include conventional as well as genetically enhanced or engineered varieties such as, for example, insect resistant as well as herbicide and disease resistant varieties.

Preferably, the stationary phase comprises an absorbent impregnated with a fluorescence indicator, preferably a silica absorbent impregnated with a fluorescence indicator active when illuminated with UV light, and wherein the illumination device comprises at least a UV light source, preferably one or more UV LED's, more preferably wherein the emission maximum of the emitted light lies in the spectral range of 265 nm to 230 nm. Most preferably, the stationary phase is impregnated with fluorescence indicator F254 (CAS: 68611-47-2).

Examples of suitable active ingredients include insecticides, fungicides, herbicides, synergists, plant growth regulators, nematicides, plant nutrients, plant fertilizers and/or biologicals.

### Step a.—Extraction of the Active Ingredient From the Seed(s)

A solvent or a combination of solvents (used herein as “extractant”) that will reproducibly extract the active ingredients from a single seed or from an aliquot of seeds is identified. This solvent or combination of solvents is termed the “extractant”. This can be different for different seeds and active ingredients and a suitable choice may be based on the solubility of the active ingredients and other factors, such as the type of the stationary phase, or the time required to remove the solvent prior to the elution process. The skilled person can readily ascertain the solvent required to extract substantially all the active ingredient(s) from the surface of a seed by routine experimentation using common laboratory solvents.

Experiments may be carried out to determine the efficacy of the extractant, in particular regarding the recovery of an active ingredient from the seed under the extraction conditions.

A representative set or aliquot of seeds is provided, or optionally a single seed may be employed, such as for instance for corn. The weight of seed is recorded using an analytical balance. Preferably, a mass of between approximately 5 and 50 g of seeds are provided, more preferably 10-30 g, most preferably 15-25 g of seeds.

Then an aliquot of a suitable extractant is added in an amount sufficient to prepare a representative test sample, and the mixture is agitated for a time adequate to substantially extract the active ingredients off the seed and into the extractant, to yield the test sample.

The test sample may optionally be decanted or filtered to remove the seeds. Alternatively, given the small volume of test sample required, simple insertion of a thin rod of inert material, such as glass or copper or a capillary tube is sufficient to separate the sample from the seeds.

In some cases, the extractant may extract from the seed other substances than the active ingredients. Examples include compounds that are soluble in the extractant, e.g., additives such as colourants, surfactants, fillers, coating agents or emulsifiers. These other substances are separated from the active ingredients by chromatography using TLC in a later step as described below.

Most preferred, however, is that the extractant comprises a polar organic solvent, preferably acetonitrile.

### Step b.—Depositing a Defined Aliquot of the Optionally Filtered Test Sample on the Starting Line of a Thin Layer Chromatographic (TLC)

The test sample aliquots are applied to the stationary phase of the TLC plate. Preferably the test sample aliquots are deposited on the “starting line” of the TLC plate (i.e. a line equidistant from the bottom edge of the TLC plate during chromatographic development). Preferably, the volumes of the test sample aliquots are deposited in a known volume onto the TLC by means of a micropipette or micro glass capillary. Preferred volumes to be deposited onto the TLC plate range from 0.1 to 100 ml (microliters), preferably from 0.5 to 50 ml (microliters), more preferably from 1 to 10 ml (microliters).

Most preferably, test sample aliquots, as well as any reference sample, are applied to the TLC plate by micro glass capillaries of a predefined volume, preferably of from 1 to 10 ml (microliters).

TLC plates are well known in the form of a plate onto which a stationary phase layer is disposed on a portion of the surface of a support layer. A TLC plate typically comprises stationary phase layer preferably a silica gel-based filler, formed on the surface of a single support sheet. The stationary phase typically also comprises an inert binder material, such as gypsum (calcium sulfate hemihydrate).

By way of non-limiting example, suitable support sheets may be made of aluminium, glass or plastic.

By way of non-limiting example, suitable stationary phases may comprise silica, aluminum oxide, cellulose or polyamides.

Commercially available TLC plates may be suitably employed, such as silica gel 60 G plates with gypsum binder available from Merck. Such plates comprise particles of silica gel having a pore size of 60 Å.

With this TLC plate, an extracted component, which is readily adsorbed by the filler layer for separation, migrates from the starting line to the silica gel-based filler layer, and in given solvent, will arrive at a certain distance from the layer, depending on its elution properties.

The sample deposited onto the TLC plate may optionally be dried before chromatographic development begins.

### Optional Step c.: Depositing an Aliquot of One or More Reference Compound(s) at a Parallel Starting Position on the Stationary Phase

Usually, reference solutions are prepared using reference material (i.e., of the targeted active ingredient) of known purity. Typically, amounts of 0.01-0.1 g are transferred to a 100 ml volumetric flask. The weight is recorded from an analytical balance to four decimal places. The volumetric flask is then filled to volume with an appropriate solvent, selected based on the solubility of the reference material. A suitable solvent for thiamethoxam and cyantraniliprole is acetonitrile.

The solution may then be manually shaken, optionally ultrasonic treatment for 10 minutes can be used, until all the reference material is dissolved, and will serve as a reference solution. An aliquot of this reference solution may then be serially diluted to prepare several standard solutions with different concentrations of active ingredient. For analysis of multiple active ingredients, a solution for each reference material may be made, which can then be combined by adding the appropriate amounts.

### Step d.—Chromatographic Development

Chromatographic development is the process of partially immersing the TLC plate into an elution solvent, termed the “mobile phase”, allowing the “mobile phase” to slowly progress up the stationary phase of the TLC plate until the solvent front has progressed sufficiently along the stationary phase to separate the active ingredients of interest into separate “spots” (areas of the stationary surface comprising the active ingredients).

The choice of the “mobile phase” may be predetermined by the active ingredient of interest. Preferably, the mobile phase, or in other words the mobile solvent phase, is selected from one or more common laboratory solvent(s) to give the active ingredient(s) a retention factor (Rf) of between 0.05 and 0.95, more preferably between 0.20 and 0.80, even more preferably of between 0.30 and 0.70 and most preferably of between 0.40 and 0.60.

Chromatographic development may be performed in a chamber. There are various chambers commercially available, differing in materials. For instance, chambers may suitably be made of glass and have glass or stainless-steel lids or be made of PTFE and have glass lids. The chambers are typically designed to allow easy insertion of both the mobile phase and the TLC plate and of a width sufficient to maintain the TLC plate in an orientation so that most of the plate is above the surface of the mobile phase. Such chambers are typically designed to allow the chamber to be fully or partially closed off from external atmosphere. This advantageously allows for high partial pressure of the mobile phase in the gas phase around the TLC plate and minimizes evaporation of the mobile phase from the surface of the active phase during chromatographic development.

### Step e

The TLC plate is removed from the TLC chamber after chromatographic development. It is preferable to dry the TLC plate after removal from the TLC chamber. The TLC plate may be dried by evaporation under ambient conditions or by heating (e.g. with a heat gun).

### Steps f. to j.—Active Ingredient Determination and Quantification

After processing in the elution chamber and drying, the TLC plate is positioned in an illumination housing according to the invention. This comprises a first housing part which comprises the illumination sources, and a holder for positioning the eluted and developed TLC plates, and a second housing portion for engaging with an imaging device comprising a camera. The housing portions are configured to optically align the camera unit, TLC plate and illumination source or sources, for optimal reproducibility and pixel resolution of the raw images of the TLC plate, and for excluding or at least limiting any incandescence of light from outside of the housing, which may influence the measurements. The portable unit is preferably battery-operated. TLC typically employs the use of a stationary phase comprising a UV fluorescence indicator, such as fluorescence indicator F254 (CAS: 68611-47-2), which fluoresces in the visible range under illumination of UV light, such as under light with a wavelength of 254 nm. The chemicals of interest either decrease the intensity of fluorescence of the indicator by quenching it, or more rarely increase the intensity of light emitted due to being fluorescent themselves. Such illumination is typically conducted in a “dark box”, which is a box configured to minimize external light sources other than the UV light source. Within the context of the present invention, where the resulting signals from the active ingredients are coloured (active ingredient(s) absorbs and/or emits light in the visible light range of 380 nm to 700 nm), the location of the active ingredient(s) on the TLC plate can be determined and the intensity of absorbed and/or emitted light may be determined by taking at least one image of the TLC plate under illumination with visible light (380-700 nm). Where the active ingredient(s) absorb light in the UV light range (10 nm to 400 nm) and emit light in the visible light range (380 nm to 700 nm), the method entails illumination with UV light comprising light with a wavelength within the range of wavelengths absorbed by the one or more active ingredient.

In a particularly preferred embodiment, the method entails using a TLC plate comprising a stationary phase, wherein the stationary phase comprises an absorbent impregnated with a fluorescence indicator and quantifying the intensity of fluorescence-quenching of each signal to estimate the amount of the one or more specific active ingredient(s). The step of quantifying the intensity of fluorescence-quenching of each signal to estimate the amount of the one or more specific active ingredient(s) comprises illuminating the TLC plate with UV light. The stationary phase is preferably a silica absorbent impregnated with a fluorescence indicator active at UV light, and wherein the illumination device comprises at least a UV light source, preferably one or more UV LED's, more preferably wherein the wavelength of the light and the emission maximum lie in the range of from 265 nm to 230 nm. Most preferably, the stationary phase is impregnated with fluorescence indicator is fluorescence indicator F254 (CAS: 68611-47-2).

A test sample typically can have a concentration of about 0.7 mg/ml of each active ingredient in the extractant.

A typical volume of 2 microlitres as test sample would require detection of amounts as small as 1.4 micrograms of active ingredient. The following TLC steps can be followed: using a TLC plate comprising a stationary phase, wherein the stationary phase comprises an absorbent impregnated with a fluorescence indicator that absorbs light in the range 230-265 nm, and quantifying the intensity of fluorescence-quenching of each signal to estimate the amount of the one or more specific active ingredient(s) under illumination of the TLC plate with UV light in the range of 230-265 nm, and measuring the intensity of fluorescence quenching in the visible light range (380 nm to 700 nm).

Technical details for method are provided in the description below.

Preferably, the imaging device comprises an electronic camera unit, preferably wherein the camera is equipped to capture light intensity in the visible light region (380 nm to 780 nm). More preferably, the imagining device is a mobile phone. Suitable mobile phones and cameras include those of an Apple iPhone XS with a camera with the following parameters: 2 MP, f/1.8, 26 mm (wide), 1/2.55″, 1.4 μm, dual pixel PDAF, OIS 12 MP, f/2.4, 52 mm (telephoto), 1/3.4″, 1.0 μm, PDAF, OIS, 2× optical zoom.

The method optionally further comprises transferring the one or more image(s) obtained in step g. to a remote processing unit and performing steps h. and i. in the remote processing unit.

The one or more image(s) obtained in step g. may optionally be transferred to a remote processing unit and performing steps h. and i. in a computer implemented application programme installed on the mobile phone.

Preferably, the method employs a detector unit configured with a recess into which a mobile phone can fit. The recess is configured to allow the mobile phone to sit on top of the detector unit and be deep enough to avoid a user accidentally pushing the mobile phone off the detector unit. The recess would be configured with a port allowing the camera of the mobile phone to face the interior of the detector unit such that a photograph of a TLC plate within the detector unit could be acquired with the mobile phone resting within the recess of the detector unit. Such a detector unit would advantageously allow the mobile phone to physically occlude external, ambient light from entering the interior of the detector unit through the port and allow the camera of the mobile phone to take a picture of a TLC plate within the detector unit. A further advantage of such a detector unit is that the distance from the camera to the TLC plates within the detector unit may be easily controlled and variance between different TLC plate photographs minimised.

Preferably, the detector unit comprises a seat configured to receive a mobile phone such that incandescent outside light is excluded, and wherein the distances and relative positions of UV-light sources, developed TLC plate and the image acquisition geometry are defined.

Preferably, the corresponding intensities of the signals are determined by comparing spots derived from a reference solution of known concentration. More preferably, the corresponding intensities of the signals are determined by comparing spots derived from a reference solution of corresponding active ingredient(s) of interest of known concentration(s).

### Visualisation and Image Capture

A benefit of the present method is that it may preferably be executed as a portable thin-layer chromatography (TLC) unit, and preferably as a portable, field-deployable thin-layer chromatography (TLC) unit, and a portable, battery-operated unit for development, illumination, and data acquisition of the TLC plates, preferably TLC plates of standard dimensions. Also, an elution chamber and an image capture housing can be dimensioned to allow for a suitable position of the TLC plate at part of the invention. This system is particularly ideal for on-site field analysis, identification and quantification of active ingredients.

Illumination and Data Acquisition: the imaging unit is preferably executed as a hand portable, battery-operated unit for development, illumination, and data acquisition of the processed TLC plates.

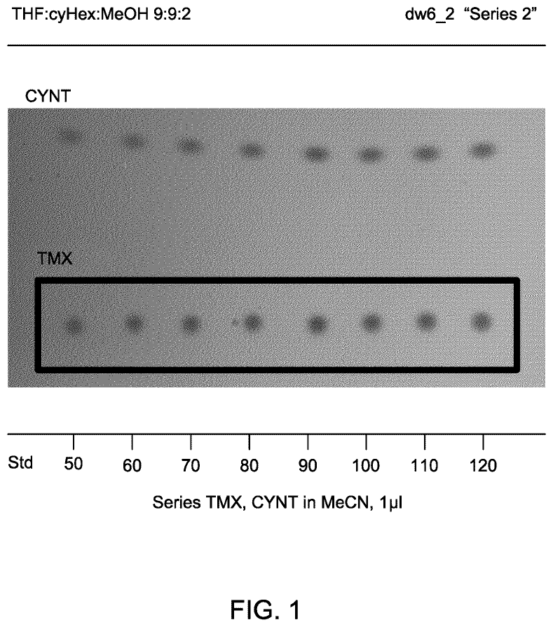

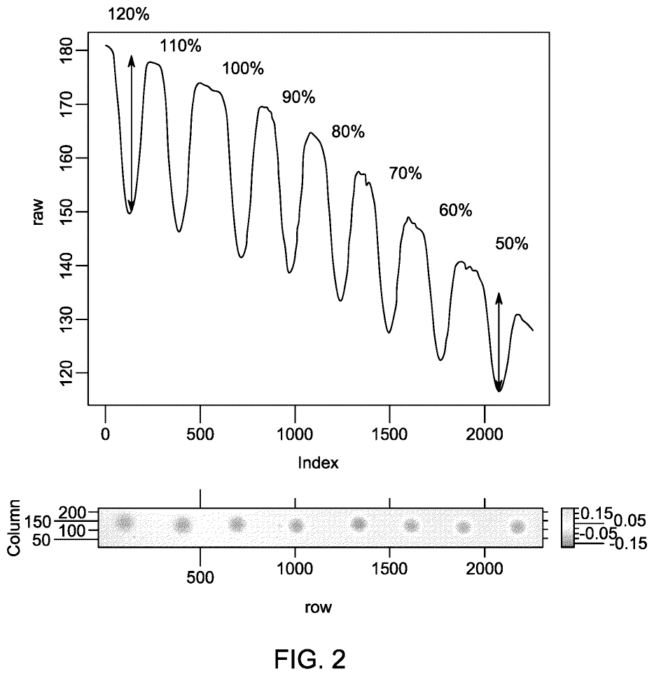

After chromatographic separation is complete, extracting quantitative information from the TLC plate requires integration of signal spot intensity (see [FIG. 2]). The separated components may be read directly as dark spots on an otherwise fluorescent surface when illuminated by UV light (sec [FIG. 1])

The camera preferably has a sufficiently high resolution, as the image data is directly recorded onto the mobile phone and can be either downloaded by a remote server for determination, or directly to a computer, or advantageously, the mobile phone used for the process may comprise software that may be used to integrate the intensity of each signal, and to determine the seed loading by comparing the signal intensities of the sample to the ones of the reference compounds included in the test.

The second housing part may advantageously be removably attached to the mobile phone in a manner that at least optically couples the housing to the resident mobile phone camera, wherein the housing is formed such that the mobile phone substantially excludes any external incandescent light to fall into the housing when a TLC plate is placed therein.

Furthermore, the housing preferably may include at least a light diffuser in the housing for providing diffuse illumination of a surface of the TLC plate disposed therein from an internal light source resident in the housing.

A particularly preferred embodiment of the invention is a field method for determining the presence and quantity of thiamethoxam and/or cyantraniliprole on one or more plant seed(s), comprising the steps of:
a. contacting the one or more plant seed(s) with an extractant to extract of thiamethoxam and/or cyantraniliprole, to yield a test sample;
b. depositing a defined aliquot of the optionally filtered test sample on the starting line of a thin layer chromatographic (TLC) plate comprising a stationary phase coated on a substrate;
c. optionally, depositing an aliquot of one or more reference compound(s), and preferably depositing an aliquot of a solution containing one or more reference compound(s), at a parallel starting position on the stationary phase; and optionally, drying the thus prepared thin layer chromatographic plate;
d. contacting the thin layer chromatographic plate with a mobile solvent phase at the lower edge of the stationary phase in an elution chamber, and allowing the sample to elute for a predetermined period of time;
e. removing the eluted thin layer chromatographic plate from the chamber, and optionally, drying the eluted thin layer chromatographic plate;
f. positioning the eluted thin layer chromatographic plate in a detector unit equipped with an illumination source and an imaging device;
g. taking at least one image of the illuminated thin layer chromatographic plate to detect the one or more signal(s) generated by the one or more active ingredient(s) and/or reference compound(s);
h. matching a retention factor (Rf) of each detected signal to an expected Rf of thiamethoxam and/or cyantraniliprole, and/or to a Rf of the reference compound(s); and
i. quantifying the intensity of fluorescence-quenching of each signal to estimate the amount of thiamethoxam and/or cyantraniliprole; and, optionally,
j. extrapolating the determined ingredient quantity to the weight or number of the one or more seed(s) subjected to extraction in step a., to obtain a seed active ingredient loading data.

Preferably, the mobile phase of step d essentially comprises cyclohexane:THF:Methanol in a volume ratio of from 10:1:1 to 1:1:10 to 1:10:1, preferably in a volume ratio of 9:9:2.

In further aspect, the invention relates to a computer implemented method for determination and quantification of active ingredient signals on an eluted TLC plate from a raw image obtained according to the method as described above, comprising the following steps:
a. detection of pixel regions in the raw image that contain the chromatographic signals;
b. assignment of the chromatographic signals to either extracted active ingredient or reference compound, and preferably to either extracted active ingredient or reference compound solution;
c. integration of the intensity of each signal in the pixel regions, the integration step c) comprising the following steps d) to g):
   a. converting RGB channels in the raw image into a luminance graph comprising a Y-channel;
   b. summing up pixel Y-intensities along the vertical dimension;
   c. detecting start- and end-points of peaks related to a signal; and
   d. integrating peak Y-intensities between peak start- and end-point; and
a. calculating active ingredient concentration by correlating the integrated peak areas of samples with the integrated peak areas of one or more reference compound(s).

Said one or more reference compound(s) in step h) can be the one provided in step c. of the method according to the invention.

In a further aspect, the invention relates to a portable apparatus for determination and quantification of an active ingredient, more preferably of an agrochemically active ingredient, extracted from a seed, and more preferably from the surface of a seed, or from a composition used or intended to be used to treat seed(s). More particularly, said portable apparatus can comprise:
a. a housing comprising a first housing portion that prevents a thin layer chromatographic plate from moving in the housing portion, such that optionally the portion of the surface does not touch any portion of the first housing portion;
b. an illumination source positioned in the first housing; and
c. a second housing portion for fitting with the first housing portion and for engaging with an imaging device such that the device permits capture of an image under illumination;
wherein the apparatus is configured to permit optimal image acquisition conditions by excluding incandescent light and by having a defined geometry (such as angle, distance and/or relative position) for both the illumination and image acquisition of a developed TLC plate

In a further embodiment, said portable apparatus can comprise:
x1) a housing preventing a thin layer chromatographic plate from moving in said housing, such that optionally the portion of the surface does not touch any portion of the first housing portion;
y1) an illumination source positioned in said housing; and
z1) an imaging device engaged with said housing such that the device permits capture of an image under illumination;
wherein the apparatus is configured to permit optimal image acquisition conditions by excluding incandescent light and by having a defined geometry (relative position, e.g. angle and distance) for both illumination and image acquisition of a developed TLC plate.

In a further embodiment, said portable apparatus can comprise:
x2) a housing comprising a first portion that prevents a thin layer chromatographic plate from moving in said housing, such that optionally the portion of the surface does not touch any portion of the first housing portion;
y2) a second housing portion for fitting with the first housing portion and wherein an illumination source is positioned; and
z2) an imaging device engaged with said second housing portion such that the device permits capture of an image under illumination;
wherein the apparatus is configured to permit optimal image acquisition conditions by excluding incandescent light and by having a defined geometry (relative position, e.g. angle and distance) for both illumination and image acquisition of a developed TLC plate.

The apparatus is advantageously portable and preferably field-deployable.

It may optionally additionally comprise a thin-layer chromatography (TLC) chromatographic development chamber and/or and a portable, battery-operated unit for development, illumination, and data acquisition of the TLC plates, preferably TLC plates of standard dimensions. This system is ideal for complete on-site field analysis, identification and quantification of active ingredients.

Illumination and Data Acquisition: the imaging unit is preferably executed as a hand portable, battery-operated unit for development, illumination, and data acquisition of the processed TLC plates. The battery-operated unit obviates the need to provide an external electricity supply (e.g. generator or mains electricity), rendering the apparatus even more suitable for field analysis in an underdeveloped agrarian setting.

The second housing part may advantageously be removably attached to the mobile phone in a manner that at least optically couples the housing to the resident mobile phone camera, wherein the housing is such formed such that the mobile phone substantially excludes any external incandescent light to fall into the housing when a TLC plate is disposed therein.

Furthermore, the housing preferably includes at least a light diffuser in the housing for providing diffuse illumination of a surface of the TLC plate disposed therein from an internal light source resident in the housing.

A further aspect of the invention relates to kit of parts, especially for field use and for other applications such as the ones mentioned in the present invention, comprising the apparatus as defined in the present description, a volume of the extractant, a volume of the mobile phase, thin layer chromatographic plates, reference standards and micro glass capillaries with a predefined capillary volume; and a mobile device comprising camera; and installed thereon, a software application.

The kit of parts may optionally comprise instructions on how to perform any of the methods described above.

A further aspect of the invention relates to a kit of parts comprising: (i) an extractant suitable for at least one active ingredient; (ii) a mobile phase suitable for chromatographically developing said at least one active ingredient; (iii) a reference standard solution of said at least one active ingredient; and (iv) a TLC plate.

The kit of parts may optionally comprise instructions on how to perform any of the methods described above. Preferably, the instructions instruct the user on how to download software onto their mobile device so as to be able to perform any of the methods above with their own mobile device.

Preferably the TLC plate has markings indicating where the aliquot of the test sample and reference sample should be deposited on the starting line, more preferably markings indicating which direction the TLC plate should be immersed into the mobile phase.

The kit of parts preferably additionally comprises micro glass capillaries of defined volume, more preferably of micro glass capillaries of between 1 and 10 μL volume, even more preferably of between 1.5 and 5 μL, most preferably of between 2 and 3 μL.

The present invention will be further explained, illustrated, and described in the following examples of systems of the present invention. The examples demonstrate the utility and/or function of the invention and help provide a full description of the invention. The examples are intended to be illustrative and not limitative of the present invention.

### EXAMPLES

Seeds were treated with Fortenza Duo, a commercially available seed treatment insecticide (commercially available from Syngenta), which comprises the insecticidal active ingredients thiamethoxam and cyantraniliprole. The mass of the seeds was accurately determined using a scale (19.9 g), and were then extracted with a known volume (38.9 g, corresponding to 49.5 mL) of acetonitrile (CH₃CN) (analytical grade from Fisher Scientific) by shaking manually for 5 minutes in a closed container, such that substantially all the active ingredients thiamethoxam and cyantraniliprole were extracted into the extractant acetonitrile. An aliquot of the extract sample (2 μL) was applied to a TLC plate (on the starting line) by disposable micro glass capillaries of defined volume (2 μL). The TLC plate was a commercially available Silica 60 G TLC plate (size: 25×100 mm) impregnated with 254 nm fluorescence indicator (from Merck). An aliquot of reference sample of a known concentration of thiamethoxam and cyantraniliprole in acetonitrile was deposited onto the TLC plate (on the same starting line, but not overlapping with the sample). The TLC plate was dried by evaporation at room temperature for 5 minutes. The TLC plate was then chromatographically developed using a mobile phase (cyclohexane:THF:MeOH, 9:9:2, by volume) in a standard glass TLC developing chamber (such as the Latch-lid™ developing chamber, commercially available from Sigma Aldrich). The TLC plate was removed from the developing chamber and allowed to dry by evaporation at room temperature for 10 minutes.

The dried TLC plate was introduced into in a detector unit equipped with an illumination source (standard, commercially available UV light source typically used for TLC analysis, λ_{MAX} at approximately 250-260 nm) and an imaging device (such as Apple iPhone XS). The mobile phone was located within a recess in the outside wall of the imaging device with its principle camera facing the interior of the detector unit through a port. The mobile phone thus located physically blocked ambient light from entering the detector unit through said port when located in the recess. The TLC plate was positioned parallel to the wall with said port. A photograph was taken of the TLC plate under illumination with UV light. The active ingredients of both the sample and the reference sample led to quenching of the fluorescence indicator, with resultant “darkness” of the spot (lower observed intensity) clearly visible in the photograph thereby obtained.

The retention factor (Rf) of each detected signal was matched to a Rf of the reference compounds (thiamethoxam and cyantraniliprole). The intensity of each signal was quantified to estimate the amount of thiamethoxam and cyantraniliprole (here quantifying the intensity of fluorescence-quenching of each signal). This was done by: a) detection of pixel regions in the raw image that contain the chromatographic spots; b) correct assignment of the chromatographic spots to the reference solution; and c) Integration of intensities of each spot (e.g. in the pixel regions detected before). The integration step comprised: d) converting RGB channels in the raw image into luminance (Y-channel); e) summing up pixel Y-intensities along vertical dimension; f) detecting start- and end-points of each peak for integration in resulting chromatogram (horizontal pixels vs Y; spots are now represented as peaks) by threshold-methods using derivatives (e.g. 2^{nd} derivative) or background subtraction; g) Integrating peak Y-intensities between the identified peak start- and end-points. The concentration of active ingredients thiamethoxam and cyantraniliprole was calculated by comparing the integrated peak areas of samples with the integrated peak areas of the reference compounds. The active ingredient loading on the seed could therefore be established from the known mass of the treated seeds and solvent volume used for extraction.

1. A method for determining the presence and quantity of one or more agrochemically active ingredient(s) on the surface of one or more plant seed(s) or in a composition used or intended to be used to treat seed(s), comprising the steps of:
a. contacting the one or more plant seed(s) or said composition, with an extractant to extract one or more active ingredient(s), to yield a test sample;
b. depositing a defined aliquot of the optionally filtered test sample on the starting line of a thin layer chromatographic (TLC) plate comprising a stationary phase coated on a substrate;
c. optionally, depositing an aliquot of one or more reference compound(s) at a parallel starting position on the stationary phase; and optionally, drying the thus prepared thin layer chromatographic plate;
d. contacting the thin layer chromatographic plate with a mobile solvent phase at the lower edge of the stationary phase in an elution chamber, and allowing the sample to elute for a predetermined period of time;
e. removing the eluted thin layer chromatographic plate from the chamber, and optionally, drying the eluted thin layer chromatographic plate;
f. positioning the eluted thin layer chromatographic plate in a detector unit equipped with an illumination source and an imaging device;
g. taking at least one image of the illuminated thin layer chromatographic plate to detect the one or more signal(s) generated by the one or more active ingredient(s) and/or reference compound(s);
h. matching a retention factor (Rf) of each detected signal to an expected Rf of a known active ingredient(s), and/or to a Rf of the reference compound(s); and
i. quantifying the intensity of each signal to estimate the amount of the one or more specific active ingredient(s), preferably quantifying the intensity of fluorescence-quenching of each signal; and, optionally,
j. extrapolating the determined ingredient quantity to the weight or number of the one or more seed(s) or of said composition, subjected to extraction in step a., to obtain an active ingredient loading data.

2. The method according to claim 1 wherein the stationary phase comprises an absorbent impregnated with a fluorescence indicator, preferably a silica absorbent impregnated with a fluorescence indicator active at UV light, and wherein the illumination device comprises at least a UV light source, preferably one or more UV LED's, more preferably wherein the wavelength of the light and the absorption maximum lie in the range of from 265 nm to 230 nm.

3. The method according to claim 1 wherein the extractant comprises a polar organic solvent, preferably acetonitrile.

4. The method according to claim 1, wherein the mobile phase essentially comprises cyclohexane:THF:Methanol in a volume ratio of from 10:1:1 to 1:1:10 to 1:10:1, preferably in a volume ratio of 9:9:2.

5. The method according to claim 1, wherein the sample aliquots are applied to the TLC plate by micro glass capillaries of a predefined volume, preferably of from 1 to 10 ml (microliters).

6. The method according to claim 1, wherein the imagining device comprises am electronic camera unit, preferably wherein the camera is equipped to capture light intensity in the visible light region between 380 nm and 700 nm.

7. The method according to claim 1, wherein the imagining device is a mobile phone.

8. The method according to claim 1, further comprising transferring the one or more image(s) obtained in step g to a remote processing unit, and performing steps h., i., and optionally, j., in the remote processing unit.

9. The method according to claim 1, further comprising transferring the one or more image(s) obtained in step g to the mobile phone, and performing steps h., i., and optionally, j., in a computer implemented application programme installed on the mobile phone.

10. The method according to claim 1, wherein the detector unit comprises a seat configured to receive a mobile phone such that incandescent outside light is excluded, and wherein the distances and relative positions of UV-light sources, developed TLC plate; and the image acquisition geometry are defined.

11. The method according to claim 1, wherein to the corresponding intensities of the signals are determined by comparing the signals to signals derived from one or more reference compound(s) of known concentrations.

12. A computer implemented method for determination and quantification of agrochemically active ingredient signals on an eluted TLC plate from a raw image obtained from the method according to claim 1, comprising the following steps:
a) detection of pixel regions in the raw image that contain the chromatographic signals;
b) assignment of the chromatographic signals to either extracted active ingredient or reference compound; and
c) integration of the intensity of each signal in the pixel regions, the integration step c) comprising the following steps d) to g):
d) converting RGB channels in the raw image into a luminance graph comprising a Y-channel;
e) summing up pixel Y-intensities along the vertical dimension;
f) detecting start- and end-points of peaks related to a signal;
g) integrating peak Y-intensities between peak start- and end-point; and
h) calculating active ingredient concentration by correlating the integrated peak areas of samples with the integrated peak areas of one or more reference compound(s).

13. A portable apparatus for determination and quantification of an agrochemically active ingredient extracted from the surface of a seed or from a composition used or intended to be used to treat seed(s), comprising:
x) a housing comprising a first housing portion that prevents a thin layer chromatographic plate from moving in the housing portion;
y) an illumination source positioned in the first housing; and
z) a second housing portion for fitting with the first housing portion and for engaging with an imaging device such that the device permits capture of an image under illumination;
wherein the apparatus is configured to permit optimal image acquisition conditions by excluding incandescent light and by having a defined geometry for both illumination and image acquisition of a developed TLC plate.

14. A portable apparatus for determination and quantification of an agrochemically active ingredient extracted from an element, comprising:
x1) a housing preventing a thin layer chromatographic plate from moving in said housing;
y1) an illumination source positioned in said housing; and
z1) an imaging device engaged with said housing such that the device permits capture of an image under illumination;
wherein the apparatus is configured to permit optimal image acquisition conditions by excluding incandescent light and by having a defined geometry for both illumination and image acquisition of a developed TLC plate.

15. A kit of parts comprising the apparatus according to claim 13, a volume of an extractant, a volume of a mobile phase, thin layer chromatographic plates, reference standards and micro glass capillaries with a predefined capillary volume; and a mobile device comprising camera; and installed thereon, a software application.
